(19) United States
(12) Patent Application Publication
(10) Pub. No.: US 2007/0093844 A1
(43) Pub. Date: Apr. 26, 2007

(54) Calcar planers for minimally invasive surgery

(76) Inventor: Donald W. Dye, Warsaw, IN (US)

Correspondence Address: ZIMMER TECHNOLOGY - BAKER & DANIELS, 111 EAST WAYNE STREET, SUITE 800, FORT WAYNE, IN, 46802, US

(21) Appl. No.: 11/248,867

(22) Filed: Oct. 12, 2005

(51) Int. Cl. A61B 17/00

(52) U.S. Cl. 606/84

(57) ABSTRACT

Calcar planers for minimally invasive surgery. The calcar planers each generally include a shaft including a power equipment interface for coupling to a power source for imparting rotary motion to the calcar planer. The shaft is connected to a cutting head via a coupling portion. The coupling portion may include a flexible coupling or a flexible segmented portion structure. Alternatively, the coupling portion may include a gear arrangement. In another embodiment, the coupling portion may include a constant velocity universal joint structure.

### BACKGROUND

1. Field of the Invention

The present invention relates to calcar planers, and, more particularly, to calcar planers for minimally invasive surgery.

2. Description of the Prior Art

During a total hip arthroplasty (THA), a surgeon typically creates an incision proximate the hip of a patient and subsequently reams a cavity in the intramedullary canal of the femur of the patient. The surgeon may then temporarily implant a rasp into the reamed cavity. The rasp includes a broach pin protruding from a proximal end of the rasp. The protruding broach pin is used as a bearing trunnion or guide pin for placement of a calcar planer. In operation, the calcar planer is inserted into the patient via the incision and mates with the broach pin of the rasp via a socket formed in the cutting head of the calcar planer. Upon mating the broach pin and the socket, the calcar planer is rotated to perform a planing of the calcar surface on the proximal femur. Once the calcar surface is sufficiently flat for the desired application, the surgeon removes the calcar planer from the patient.

Conventional calcar planers include a straight, rigid shaft directly connecting the cutting head to a rotation-imparting power source. In some circumstances involving minimally invasive surgery, a direct access to the broach pin via the incision may not be available due to the reduced size and/or placement of the incision. The rigid construction of a conventional calcar planer could potentially require a surgeon to enlarge the entry incision to prevent the shaft of the calcar planer from impinging on the edge of the incision.

### SUMMARY

The present invention provides calcar planers for minimally invasive surgery. A calcar planer in accordance with the present invention generally includes a shaft having a longitudinal axis including a power equipment interface for coupling to a power source for imparting rotary motion to the calcar planer. The shaft is connected via a coupling portion to a cutting head having a longitudinal axis. The coupling portion may include a flexible coupling or a flexible segmented structure. Alternatively, the coupling portion may include a gear arrangement. In another embodiment, the coupling portion may include a constant velocity universal joint (U-joint) structure. In each of the foregoing embodiments, the cutting head longitudinal axis of the calcar planer of the present invention is either selectively or fixedly non-coaxial with the shaft longitudinal axis.

When the cutting head longitudinal axis is non-coaxial with the shaft longitudinal axis, torque is advantageously transmitted from the shaft to the cutting head via the coupling portion. The coupling portion advantageously permits transmission of rotational torque even when the shaft is not aligned with the cutting head. When misaligned, the power source transmits torque to the shaft which, in turn, transmits rotational motion to the coupling portion. The coupling portion transmits the rotational motion around the angle formed by the shaft and the cutting head to the cutting head. The coupling portion advantageously permits a surgeon to angularly move the shaft about the cutting head longitudinal axis while still simultaneously transmitting torque from the shaft to the cutting head.

In one form thereof, the present invention provides a calcar planer for use in planing a calcar surface of a bone including a shaft including a first longitudinal axis; a cutting head including a second longitudinal axis; and a torque transmitting coupler connecting the shaft and the cutting head, the coupler transferring torque between the shaft and the cutting head when the first and second axes are coaxially aligned and when the first and second axes are not coaxially aligned.

In another form thereof, the present invention provides a calcar planer for use in planning a calcar surface of a bone including a shaft including a first longitudinal axis; a cutting head including a second longitudinal axis; and torque transmission coupler means connecting the shaft and the cutting head for transferring torque between the shaft and the cutting head while concurrently allowing axial misalignment between the first and second axes.

In yet another form thereof, the present invention provides a calcar planer for use in planing a calcar surface of a bone including a shaft including a first longitudinal axis; a cutting head including a second longitudinal axis; a torque transmitting coupler connecting the shaft and the cutting head, the coupler transferring torque between the shaft and the cutting head when the first and second axes are coaxially aligned and when the first and second axes are not coaxially aligned; a flexible sheath disposed around the torque transmitting coupler; and a handle, the handle connected to the calcar planer proximate the cutting head.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1A is a perspective view of an exemplary calcar planer of the present invention;

FIG. 1B is another perspective view of the calcar planer of FIG. 1A;

FIG. 1C is a perspective view of the calcar planer of FIG. 1A, additionally showing a handle coupled to the planer;

FIG. 1D is a fragmentary perspective view of an alternative embodiment of a calcar planer of the present invention;

FIG. 2A is a perspective view of another alternative embodiment calcar planer of the present invention;

FIG. 2B is an enlarged view of a portion of yet still another embodiment calcar planer, further illustrating the gear set in a cutaway portion of the calcar planer;

FIG. 2C is a perspective view of the calcar planer of FIG. 2A, additionally showing a handle coupled to the planer;

FIG. 3A is a perspective view of another alternative embodiment calcar planer of the present invention;

FIG. 3B is a close-up view of a portion of the calcar planer of FIG. 3A;

FIG. 3C is a perspective view of the calcar planer of FIG. 3A, additionally showing a handle coupled to the planer;

FIG. 3D is an enlarged view of a portion of the calcar planer of FIG. 3C; and

FIG. 4 is a perspective view of the calcar planer of FIG. 1A shown in operational relationship with a calcar surface of a femur.

Corresponding reference characters indicate corresponding parts throughout the several views. Although the drawings represent embodiments of the present invention, the drawings are not necessarily to scale and certain features may be exaggerated in order to better illustrate and explain the present invention. The exemplifications set out herein illustrate embodiments of the invention, and such exemplifications are not to be construed as limiting the scope of the invention in any manner.

### DETAILED DESCRIPTION

The embodiments disclosed below are not intended to be exhaustive or limit the invention to the precise forms disclosed in the following detailed description. Rather, the embodiments are chosen and described so that others skilled in the art may utilize their teachings.

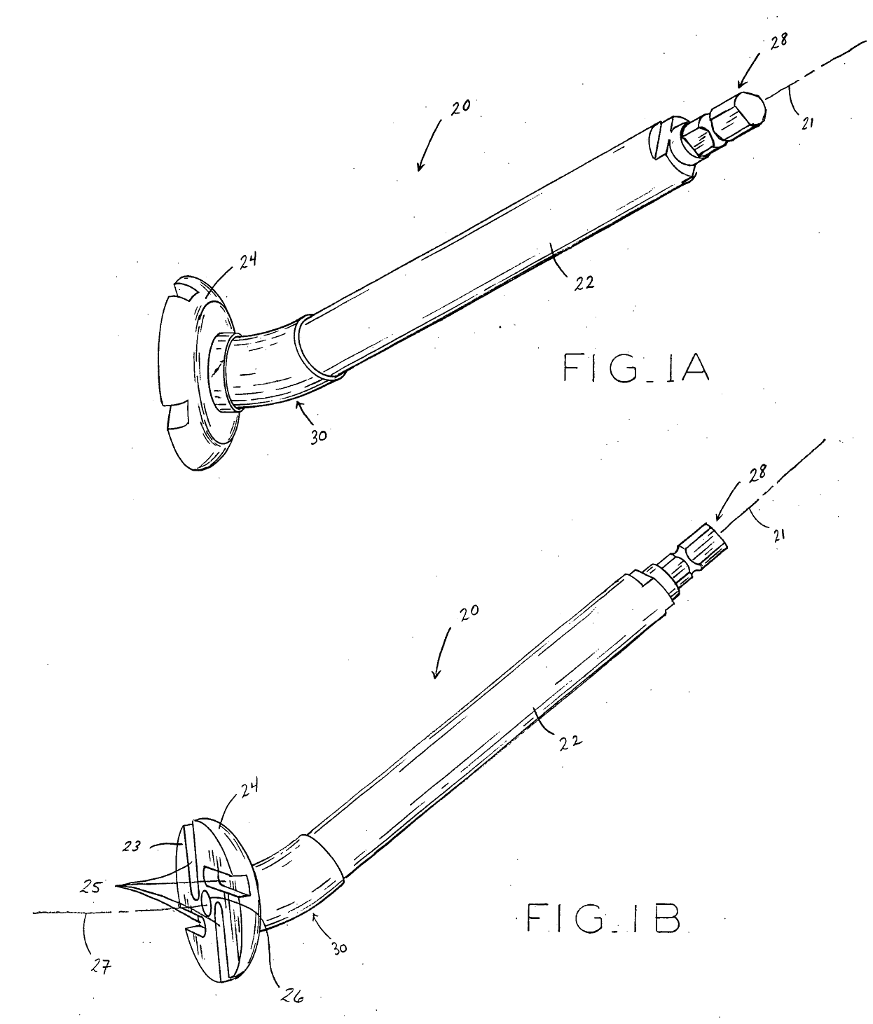

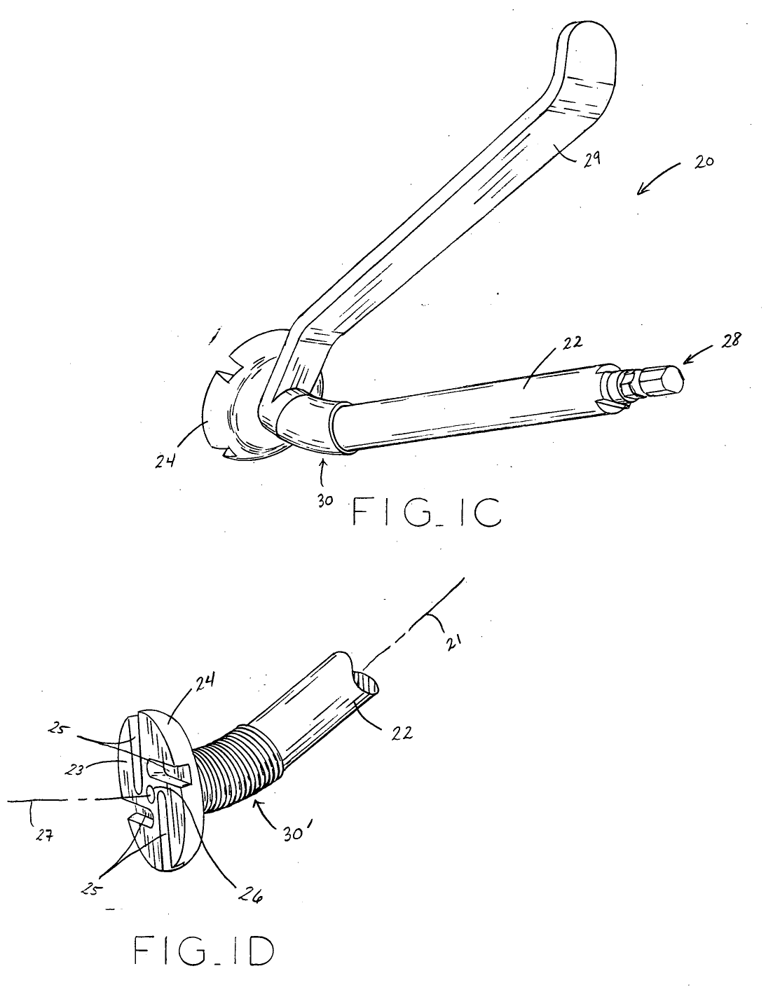

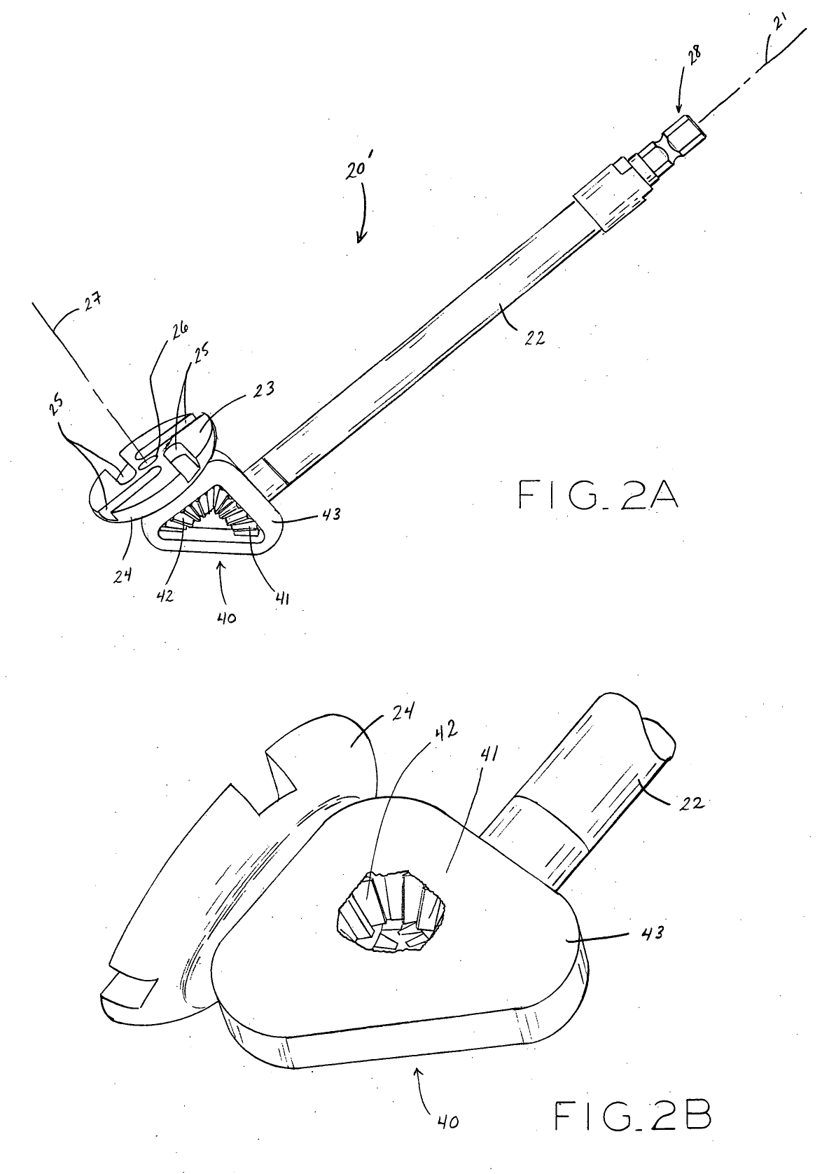

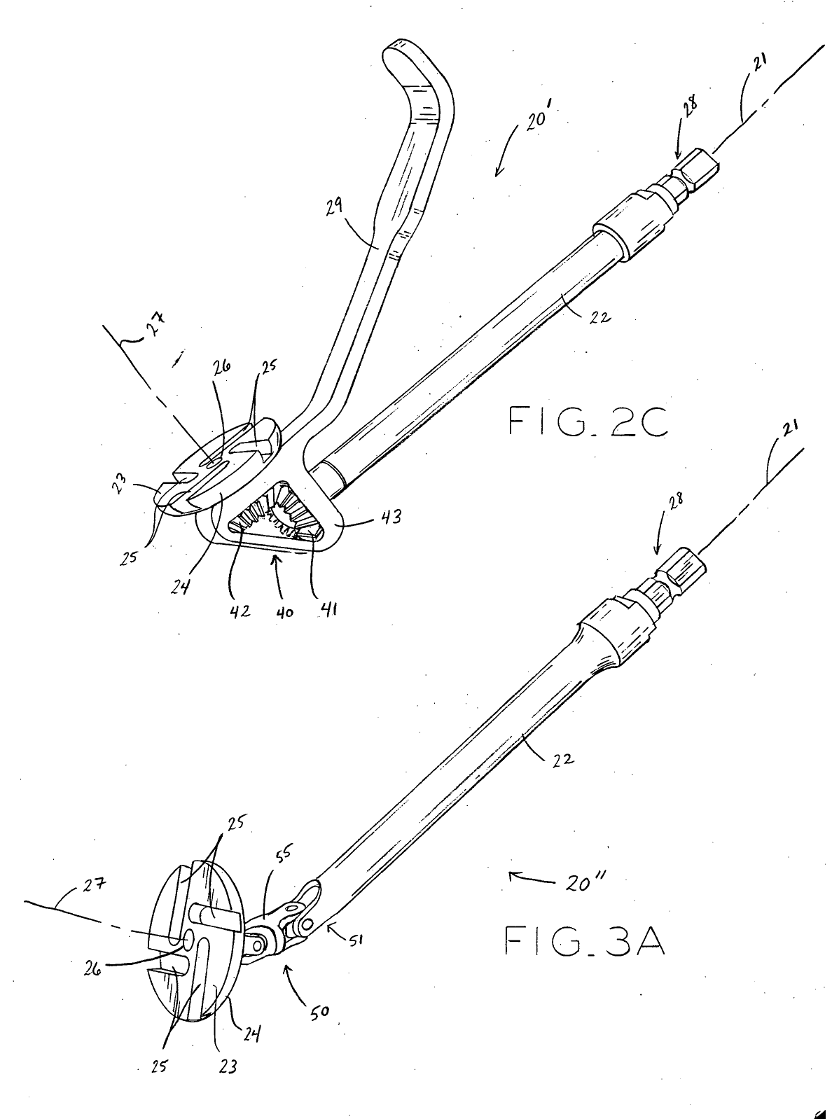

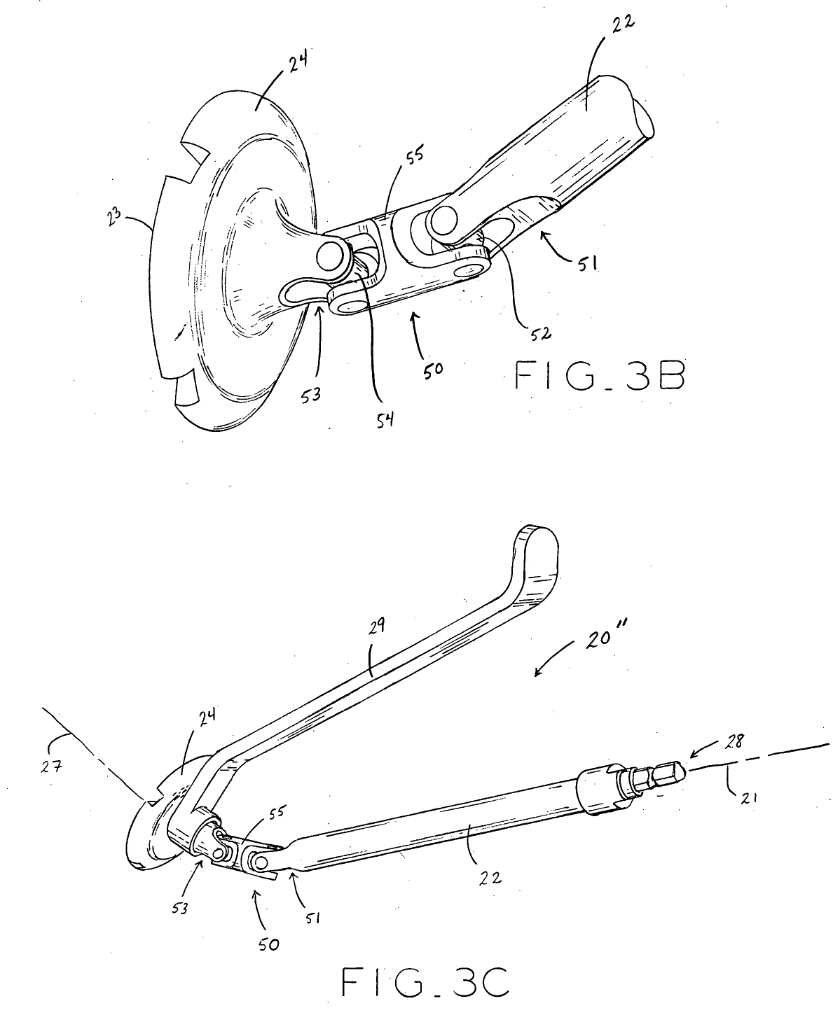

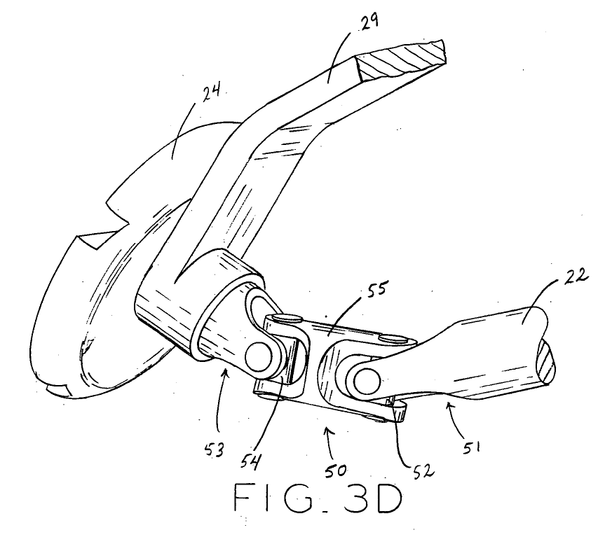

In general, the present invention provides calcar planers for minimally invasive surgery. As illustrated in FIGS. 1A-1D, 2A-2C, and 3A-3D, calcar planers 20, 20′, and 20″, respectively, each generally include shaft 22 including power equipment interface 28 for coupling to a power source (not shown) for imparting rotary motion to calcar planers 20, 20′, and 20″. Shaft 22 is connected to cutting head 24 via a coupling portion. The coupling portion may include flexible coupling 30 (FIGS. 1A-1C) or flexible segmented portion 30′(FIG. 1D). Alternatively, the coupling portion may include gear arrangement 40 (FIGS. 2A-2C). In another embodiment, the coupling portion may include constant velocity U-joint 50 (FIGS. 3A-3D) or U-joint 50′(not shown).

When cutting head longitudinal axis 27 is non-coaxial with shaft longitudinal axis 21, torque is advantageously transmitted from shaft 22 to cutting head 24 via the coupling portion. The coupling portion advantageously permits transmission of rotational torque even when shaft 22 is not aligned with cutting head 24. When misaligned, the power source transmits torque to shaft 22 which, in turn, transmits rotational motion to the coupling portion. The coupling portion transmits the rotational motion around the angle formed by shaft 22 and cutting head 24 to cutting head 24. The coupling portion advantageously permits a surgeon to angularly move shaft 22 about cutting head longitudinal axis 27 while still simultaneously transmitting torque from shaft 22 to cutting head 24.

Flexible Coupling Embodiment

Referring now to FIG. 1A, calcar planer 20 includes shaft 22 and cutting head 24 coupled together via coupling portion or flexible coupling 30. Shaft 22 includes power equipment interface 28 configured to permit shaft 22 to be coupled to a power source (not shown), such as a rotary drill, for imparting rotary motion to calcar planer 20 during use. Shaft 22 also includes longitudinal axis 21 extending along a length thereof. As shown in FIG. 1B, cutting head 24 includes cutting surface 23 having a plurality of cutting head teeth 25 and cutting head socket 26. Cutting head teeth 25 are arranged on cutting surface 23 along chord lines of the circle defined by cutting surface 23. Each chord line along which each cutting head tooth 25 is arranged is perpendicular to an adjacent cutting head tooth 25. Cutting head 24 also includes longitudinal axis 27 which extends perpendicular to the planar surface which includes cutting surface 23.

In one embodiment, flexible coupling 30 may be formed of a material such that the material resumes its original shape when a deforming force is removed and such that the material provides torsional rigidity to calcar planer 20. The material constituting flexible coupling 30 may be such as to permit longitudinal axis 27 of cutting head 24 to be selectively moved from coaxial alignment into non-coaxial alignment with longitudinal axis 21 of shaft 22 at the discretion of the surgeon, advantageously permitting shaft 22 to be angularly moved about longitudinal axis 27 while still simultaneously transmitting torque from shaft 22 to cutting head 24. Flexible coupling 30 may be formed of any material which provides a spring-type force which keeps longitudinal axis 21 of shaft 22 in straight alignment with longitudinal axis 27 of cutting head 24 without any bending force applied thereto, and facilitates the return to a straight alignment between longitudinal axes 21 and 27 after a bending force has been removed. Such material may include a vulcanized rubber material, an elastomer, e.g., rubber, a polymer material, polytetrafluoroethylene (PTFE), or polyethylene. Torque may be transmitted via flexible coupling 30 without a supporting structure therein if flexible coupling 30 is formed of a suitable material, e.g., vulcanized rubber. Alternatively, flexible coupling 30 may be formed as a cable with sufficient flexibility and constructed of a shape-memory metal alloy, e.g., nitinol, with a sheath formed of any of the above-listed materials which surrounds the flexible cable. Such an exterior sheath prevents any blood, tissue, or other bodily waste from interfering with the workings of the internal mechanism.

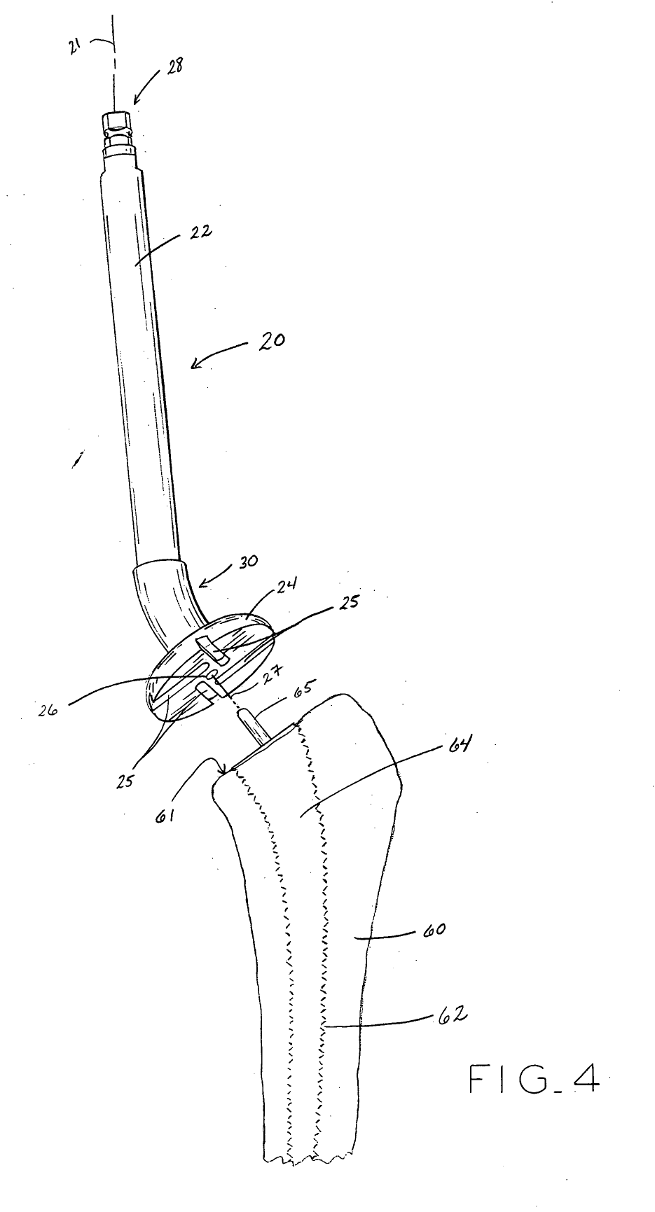

In one embodiment, as shown in FIG. 1C, calcar planer 20 may include handle 29. Handle 29 may be attached on any portion of calcar planer 20, but is shown attached between flexible coupling 30 and cutting head 24 in FIG. 1C. Handle 29 facilitates the surgeon's ability to control cutting head 24 during operation and to accurately place cutting head socket 26 onto broach pin 65 (FIG. 4), as discussed below. Handle 29 includes an internal bushing (not shown) whereby calcar planer 20 may rotate in the bushing and handle 29 does not rotate with calcar planer 20. Handle 29 advantageously provides enhanced control of the torque reaction resulting from rotation of calcar planer 20.

In an alternative embodiment shown in FIG. 1D, calcar planer 20 may include flexible segmented portion 30′which couples shaft 22 and cutting head 24. Flexible segmented portion 30′ may take the form of a flexible accordion-type structure or a bellows-type structure and may be constructed with a pleated, expandable material which is able to be expanded and contracted as well as manipulated to form a flexible, curved shape. As similarly described above with respect to flexible coupling 30, flexible segmented portion 30′ advantageously permits a surgeon to modify the orientation of shaft 22 with respect to cutting head 24 while transmitting rotational torque from shaft 22 to cutting head 24. Such modification may make longitudinal axis 27 of cutting head 24 non-coaxial with longitudinal axis 21 of shaft 22. Flexible segmented portion 30′ may be constructed of a plastic or polymer material, a metal alloy, or a woven fabric or textile.

Gear Arrangement Embodiment

Referring now to FIG. 2A, calcar planer 20′ includes shaft 22 and cutting head 24 coupled together via a coupling portion, shown as a gear arrangement 40. Gear arrangement 40 may include first gear 41 attached through gear arrangement housing 43 to shaft 22 and second gear 42 attached through gear arrangement housing 43 to cutting head 24. The connections of first gear 41 and second gear 42 to shaft 22 and cutting head 24, respectively, through gear arrangement housing 43 may include internal bushings to facilitate transmittal of rotary motion to first gear 41 from shaft 22 and cutting head 24 from second gear 42. First gear 41 and second gear 42 are in meshing engagement to transmit rotational motion from shaft 22 to cutting head 24. Upon rotation of shaft 22, the teeth of first gear 41 rotate and matingly engage the teeth of second gear 42. Upon engagement with the rotating teeth of first gear 41, the teeth of second gear 42 rotate and cause cutting head 24 to rotate.

Gear arrangement housing 43 houses first gear 41 and second gear 42 and may be formed out of any suitable biocompatible material. In one embodiment as shown in FIG. 2B, gear arrangement housing 43 completely encapsulates first gear 41 and second gear 42 in a sealed housing. FIG. 2B shows a cutaway portion revealing first gear 41 and second gear 42. The sealed housing prevents any wound debris from entering gear arrangement housing 43 which may obstruct first gear 41 and second gear 42. In the sealed housing arrangement, the connections of first gear 41 and second gear 42 to shaft 22 and cutting head 24, respectively, through gear arrangement housing 43 may also be sealed with, for example, gaskets.

Referring again to FIG. 2A, in one embodiment, gear arrangement 40 disposes longitudinal axis 21 of shaft 22 perpendicular to longitudinal axis 27 of cutting head 24. Advantageously, such a configuration allows first gear 41 and second gear 42 to be cost-effectively cut at a 45° bevel to provide a fixed, 90° power transmission. Alternatively, first gear 41 and second gear 42 may be cut such as to provide any degree of power transmission desired by an end user of calcar planer 20′. Gears 41 and 42 are cut at approximately ½ of the desired angle between longitudinal axis 21 of shaft 22 and longitudinal axis 27 of cutting head 24, for example, gears 41 and 42 may be cut at a 67.5° angle to allow shaft 22 to be at a 135° angle with respect to cutting head 24.

In one embodiment, as shown in FIG. 2C, gear arrangement housing 43 includes handle 29 extending therefrom. Handle 29 may be integrally formed with housing 43 or handle 29 may be attached with fasteners (not shown) if handle 29 is constructed as a separate piece. Alternatively, handle 29 may be attached to shaft 22 immediately proximal to housing 43 similar to the attachment of handle 29 to calcar planer 20, as described above, or handle 29 may be attached to cutting head 24 in a similar manner.

Constant Velocity U-Joint Embodiment

A universal joint (U-joint) is a flexible double-pivoted joint that allows driving power to be carried through two shafts that are at an angle to each other. A U-joint consists of two Y-shaped yokes and a cross-shaped member called the spider. Ordinary U-joints cause a change in speed between a driving shaft and a driven shaft whenever the U-joint operates at an angle. As the operating angle of the U-joint increases, the speed of the driven shaft varies more and more during each revolution. The greater the operating angle, the greater the variation in speed of the driven shaft and the greater the vibration produced.

The driven shaft still turns at the same number of revolutions per minute as the driving shaft, but because of the geometry of a U-joint, the speed of the driven shaft alternately increases (accelerates) and decreases (decelerates) four times every revolution, thereby causing vibration of the driven shaft. The speed changes are not great when the angle is less than a few degrees, but as the operating angle of the U-joint increases, so do the cyclic vibrations of the driven shaft as well as the back and forth motion in the U-joint itself.

To combat the negative effects of an ordinary U-joint, a second U-joint can be used which is phased in line with respect to the first U-joint to form a constant velocity U-joint. The second U-joint cancels out the changes in output velocity caused by the first U-joint, but only so long as both U-joints operate at identical angles. Thus, no matter what the angle between the first U-joint and the second U-joint, there are no changes in speed of the driven shaft.

Referring now to FIG. 3A, calcar planer 20″ includes shaft 22 and cutting head 24 coupled together via coupling portion or constant velocity universal joint (U-joint) 50. As shown in FIG. 3B, shaft 22 may include U-portion or yoke 51 located at a distal end thereof and cutting head 24 may include U-portion or yoke 53 located opposite cutting surface 23 on a proximal side of cutting head 24. U-portion 51 and U-portion 53 are coupled together via U-joint coupler 55 and secured thereto via spiders 52 and 54, respectively. In an alternative embodiment, the coupling portion comprises U-joint 50′ (not shown) wherein U-joint coupler 55 is absent and U-portion 51 is directly connected to U-portion 53 via a spider.

Constant velocity U-joint 50, as shown in FIGS. 3A and 3B, transmits rotational motion from shaft 22 to cutting head 24 at a constant velocity. Upon rotation of shaft 22, U-portion 51 transmits elliptical rotation to U-joint coupler 55. Upon rotation across the major axes of the ellipse, the rotational velocity is very high. In contrast, upon rotation across the minor axes of the ellipse, the rotational velocity is very low. To compensate and achieve constant velocity rotation, the interaction of U-joint coupler 55 with U-portion 53 of cutting head 24 forces cutting head 24 to rotate at a constant velocity because during the rotation across the major axes of the ellipse by U-portion 51, U-portion 53 is also rotating across the major axes of its ellipse, thereby nullifying any speed change provided by rotation of U-portion 51. Similarly, during the rotation across the minor axes of the ellipse by U-portion 51, U-portion 53 is also rotating across the minor axes of its ellipse, thereby nullifying any speed change caused by rotation of U-portion 51. The interaction and configuration of constant velocity U-joint 50 transmits a constant velocity rotational motion from shaft 22 to cutting head 24.

In an alternative embodiment, as shown in FIGS. 3C and 3D, calcar planer 20″ may include handle 29 attached between constant velocity U-joint 50 and cutting head 24 similar to handle 29, as described above with respect to calcar planer 20 in FIG. 1C.

Flexible Coupling Combined with U-Joint Embodiment

In another embodiment, flexible coupling 30, as shown in FIGS. 1A-1C, or flexible segmented portion 30′, as shown in FIG. 1D, may be combined with constant velocity U-joint 50, as shown in FIGS. 3A-3D. In this embodiment, constant velocity U-joint 50 may be encompassed within flexible coupling 30 or flexible segmented portion 30′, thereby providing a shielding effect to constant velocity U-joint 50 from any wound debris while simultaneously enhancing the ability to make longitudinal axis 21 of shaft 22 selectively non-coaxial with longitudinal axis 27 of cutting head 24. Flexible coupling 30 or flexible segmented portion 30′ may provide a flexible sheath, i.e., a protective and enveloping covering or structure, which may be positioned around constant velocity U-joint 50 or U-joint 50′ (not shown).

Method of Operation

Referring now to FIG. 4, during a total hip arthroplasty (THA), a surgeon creates an incision (not shown) proximate the hip of a patient (not shown) and subsequently reams a cavity in intramedullary canal 62 of femur 60 of the patient. The surgeon may then temporarily implant rasp 64 into the reamed cavity. Rasp 64 includes broach pin 65 protruding from a proximal end of rasp 64. The protruding broach pin 65 is used as a bearing trunnion or guide pin for placement of calcar planer 20. In operation, calcar planer 20 is inserted into the patient via the incision and mates with broach pin 65 of rasp 64 via cutting head socket 26 formed in cutting head 24 of calcar planer 20. Upon mating broach pin 65 and cutting head socket 26, calcar planer 20 is rotated to perform a planing of calcar surface 61 on the proximal end of femur 60. Once calcar surface 61 is sufficiently flat for the desired application, the surgeon removes calcar planer 20 from the patient.

During insertion of calcar planer 20, flexible coupling 30 permits efficient access to broach pin 65 with a minimally invasive incision. Due to the minimally invasive incision, the surgeon has very little space to manipulate calcar planer 20 to mate broach pin 65 with cutting head socket 26. Flexible coupling 30 permits a surgeon to control the orientation of longitudinal axis 27 of cutting head 24 and longitudinal axis 21 of shaft 22 and place axis 27 and axis 21 in a non-coaxial arrangement, as shown in FIG. 4. Such selective flexibility permits a surgeon to maintain the original, minimal size of the minimally invasive incision without having to enlarge the incision to prevent impingement of shaft 22 on the edge of the incision while guiding cutting head socket 26 into mating engagement with broach pin 65. During rotation, flexible coupling 30 permits the surgeon to maintain calcar planer 20 in an arrangement wherein axis 27 and axis 21 are in a non-coaxial arrangement which again permits the surgeon to maintain the original size of the incision without being required to enlarge the incision to prevent impingement of rotating shaft 22 on the edge of the incision. Such arrangement is facilitated through the use of handle 29 (FIG. 1C) which not only helps maintain the non-coaxial arrangement but also helps the surgeon compensate for the torque reaction of calcar planer 20 upon rotation.

While this invention has been described as having exemplary designs, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A calcar planer for use in planing a calcar surface of a bone, comprising:
a shaft including a first longitudinal axis;
a cutting head including a second longitudinal axis; and
a torque transmitting coupler connecting said shaft and said cutting head, said coupler transferring torque between said shaft and said cutting head when said first and second axes are coaxially aligned and when said first and second axes are not coaxially aligned.

2. The calcar planer of claim 1, wherein said coupler comprises a flexible coupler which enables said first and second axes to be selectively not coaxially aligned.

3. The calcar planer of claim 1, wherein said coupler comprises a constant velocity universal joint, said constant velocity universal joint transmitting constant rotational velocity from said shaft to said cutting head when said first and second axes are coaxially aligned and when said first and second axes are not coaxially aligned.

4. The calcar planer of claim 3, wherein said coupler further comprises a flexible sheath disposed around said constant velocity universal joint.

5. The calcar planer of claim 1, wherein said coupler comprises a gear set including a first gear and a second gear, said first gear in meshing engagement with said second gear, said first gear connected to said shaft, and said second gear connected to said cutting head.

6. The calcar planer of claim 5, wherein said gear set is housed within a substantially enclosed housing portion.

7. The calcar planer of claim 1, further comprising a handle, said handle connected to the calcar planer proximate said coupler.

8. A calcar planer for use in planning a calcar surface of a bone, comprising:
a shaft including a first longitudinal axis;
a cutting head including a second longitudinal axis; and
torque transmission coupler means connecting said shaft and said cutting head for transferring torque between said shaft and said cutting head while concurrently allowing axial misalignment between said first and second axes.

9. The calcar planer of claim 8, wherein said coupler means comprises a flexible coupler which enables said first and second axes to be selectively not coaxially aligned while still transferring torque between said shaft and said cutting head.

10. The calcar planer of claim 8, wherein said coupler means comprises a constant velocity universal joint, said constant velocity universal joint transmitting constant rotational velocity from said shaft to said cutting head while said first and second axes are not coaxially aligned.

11. The calcar planer of claim 10, wherein said coupler means further comprises a flexible sheath disposed around said constant velocity universal joint.

12. The calcar planer of claim 8, wherein said coupler means comprises a gear set including a first gear and a second gear, said first gear in meshing engagement with said second gear, said first gear connected to said shaft, and said second gear connected to said cutting head, said gear set transmitting torque from said shaft to said cutting head through meshing engagement of said first gear with said second gear.

13. The calcar planer of claim 12, wherein said gear set is housed within a substantially enclosed housing portion.

14. The calcar planer of claim 8, further comprising a handle, said handle connected to the calcar planer proximate said coupler means.

15. A calcar planer for use in planing a calcar surface of a bone, comprising:
a shaft including a first longitudinal axis;
a cutting head including a second longitudinal axis;
a torque transmitting coupler connecting said shaft and said cutting head, said coupler transferring torque between said shaft and said cutting head when said first and second axes are coaxially aligned and when said first and second axes are not coaxially aligned;
a flexible sheath disposed around said torque transmitting coupler; and
a handle, said handle connected to the calcar planer proximate said coupler.

16. The calcar planer of claim 15, wherein said coupler comprises a constant velocity universal joint, said constant velocity universal joint transmitting constant rotational velocity from said shaft to said cutting head when said first and second axes are coaxially aligned and when said first and second axes are not coaxially aligned.

17. The calcar planer of claim 15, wherein said coupler comprises a flexible cable.

18. The calcar planer of claim 15, wherein said coupler comprises a gear set including a first gear and a second gear, said first gear in meshing engagement with said second gear, said first gear connected to said shaft, and said second gear connected to said cutting head, said gear set transmitting torque from said shaft to said cutting head through meshing engagement of said first gear with said second gear.
